(19) United States
(12) Patent Application Publication
(10) Pub. No.: US 2024/0005077 A1
(43) Pub. Date: Jan. 4, 2024

(54) METHOD OF DESIGNING THIN FILM TRANSISTOR

(71) Applicant: Institute of Microelectronics, Chinese Academy of Sciences, Beijing (CN)

(72) Inventors: Nianduan Lu, Beijing (CN); Ling Li, Beijing (CN); Wenfeng Jiang, Beijing (CN); Di GENG, Beijing (CN); Jiawei Wang, Beijing (CN); Ming Liu, Beijing (CN)

(21) Appl. No.: 18/250,461

(22) PCT Filed: Oct. 30, 2020
(86) PCT No.: PCT/CN2020/125115

§ 371 Date: Apr. 25, 2023

(51) Int. Cl. G06F 30/39; G16C 60/00

(52) U.S. Cl. CPC G06F 30/39; G16C 60/00; H01L 29/786

(57) ABSTRACT

A method of designing a thin film transistor device, including: calculating characteristic parameters of searched materials; screening the materials according to a characteristic parameter threshold to obtain first active layer materials; simulating the first active layer material as an active layer material in a thin film transistor device model to obtain a device characteristic of the thin film transistor device; screening the first active layer materials according to a device characteristic threshold to obtain second active layer materials; taking the second active layer material as the active layer material of the thin film transistor device to perform an experiment; and selecting another second active layer material to perform the experiment once again when an experiment result does not meet a preset requirement, and a design of the thin film transistor device is completed until the experiment result meets the preset requirement.

### TECHNICAL FIELD

The present disclosure relates to a field of semiconductor transistor device, and in particular, to a method of designing a thin film transistor.

### BACKGROUND

As a useful supplement to the field effect transistor, the thin film transistor has been widely applied in display, sensing and other fields in recent years. Compared with the field effect transistor, various semiconductor materials may be used to manufacture the thin film transistor, and the manufacturing process thereof is relatively simple. Relatively cheap large-area spin coating and printing may be used, and thus the manufacturing cost is relatively low. In addition, as the thin film material may be manufactured at a lower temperature, a material with a poor heat resistance (for example, a substrate such as plastic paper, etc.) may be selected to manufacture a light-weight, tough and bendable electronic device. However, as a wide variety of materials may be used to manufacture the thin film transistor device, there are still many technical problems in reliability, current characteristics and durability of thin film transistor devices made of different materials. As a result, there is no system of thin film transistor devices that may completely replace the existing technology to occupy a dominant position in the market.

At present, people generally use experimental methods to try to improve the comprehensive performance of the thin film transistor device from aspects of material, structure, composition, testing and the like. However, it not only consumes a lot of time and costs to carry out researches through experimental methods, but also the research process is relatively slow. Compared with the experiments, using theoretical methods to study the thin film transistor device may not only predict, analyze, and optimize device characteristics, but also greatly shorten the research process. However, the structure of the thin film transistor is simple, but factors that affect the characteristics thereof are diverse. It is difficult to fully describe the characteristics of the thin film transistor device with current experimental methods and theoretical methods. Therefore, in order to accelerate the development of the thin film transistor and its application in display technology, it is of great significance to develop an effective method to design the thin film transistor device.

### SUMMARY

One of the main objectives of the present disclosure is to provide a method of designing a thin film transistor device.

Specifically, the present disclosure provides a method of designing a thin film transistor device, comprising: calculating characteristic parameters of searched materials; screening the materials according to a characteristic parameter threshold to obtain first active layer materials; simulating the first active layer material as an active layer material in a thin film transistor device model to obtain a device characteristic of the thin film transistor device; screening the first active layer materials according to a device characteristic threshold to obtain second active layer materials; taking the second active layer material as the active layer material of the thin film transistor device to perform an experiment; and selecting another second active layer material to perform the experiment once again when an experiment result does not meet a preset requirement, and a design of the thin film transistor device is completed until the experiment result meets the preset requirement.

### BRIEF DESCRIPTION OF THE DRAWINGS

FIG. 1 is a flowchart of a method of designing a thin film transistor device of the present disclosure.

FIG. 2 is a schematic flowchart of a method of designing a thin film transistor device in the embodiments of the present disclosure.

### DETAILED DESCRIPTION OF EMBODIMENTS

In order to make the objectives, technical solutions and advantages of the present disclosure clearer, the present disclosure will be further described in detail below in combination with specific embodiments with reference to the accompanying drawings.

According to analyses of the current situation of the related research field, the present disclosure proposes a method of designing a thin film transistor device based on high throughput integration and first principle calculation method. Such method is simple to operate and may be widely applied in design of various thin film transistor devices of different materials and structures.

Generally speaking, the high throughput integrated calculation refers to a theoretical prediction of potential new structures and new formulas by using a method of submitting a large number of calculating tasks at one time through element substitution, high throughput screening, structure optimization, and related property calculations, etc. The design of the thin film transistor device having a high performance integrates the selection of initial materials, composition design and screening, device structure design and optimization, performance prediction and feedback, etc. of the thin film transistor device for a comprehensive consideration, and then performs a control using an automatic calculation module, so as to achieve an automated design process of the thin film transistor device and finally design a thin film transistor device having a superior comprehensive performance.

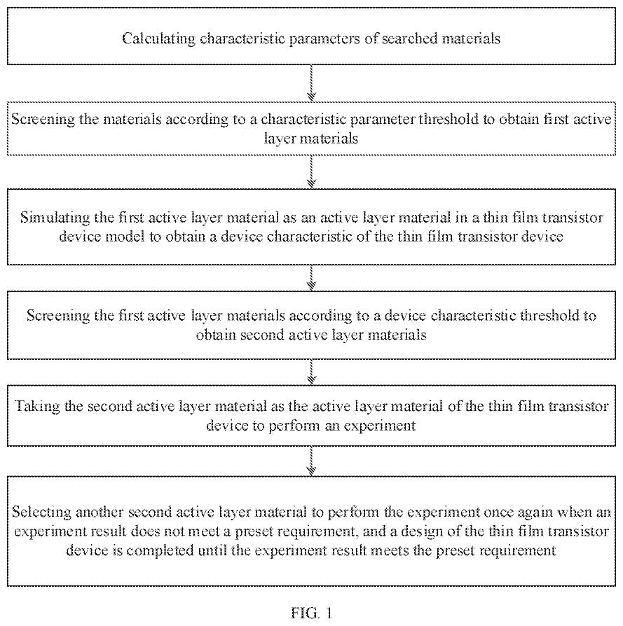

As shown in FIG. 1, the present disclosure provides a method of designing a thin film transistor device, including:
calculating characteristic parameters of searched materials;
screening the materials according to a characteristic parameter threshold to obtain first active layer materials;
simulating the first active layer material as an active layer material in a thin film transistor device model to obtain a device characteristic of the thin film transistor device;
screening the first active layer materials according to a device characteristic threshold to obtain second active layer materials;
taking the second active layer material as the active layer material of the thin film transistor device to perform an experiment; and
selecting another second active layer material to perform the experiment once again when an experiment result does not meet a preset requirement, and a design of the thin film transistor device is completed until the experiment result meets the preset requirement.

In some embodiments of the present disclosure, the characteristic parameter includes at least one of energy band structure, band gap, Schottky barrier, work function, and intermediate phase.

In some embodiments of the present disclosure, the characteristic parameter threshold includes at least one of band gap threshold, Schottky barrier threshold, work function threshold or intermediate phase threshold.

In some embodiments of the present disclosure, the band gap threshold is 0.5 to 3 eV.

In some embodiments of the present disclosure, the Schottky barrier threshold is 0.1 to 2 eV

In some embodiments of the present disclosure, the work function threshold is 2.5 to 5.5 eV.

In some embodiments of the present disclosure, the intermediate phase threshold is 1 to 4.

In some embodiments of the present disclosure, the simulating step specifically includes simulating a transfer curve (transfer characteristic) and an output curve (output characteristic) of the thin film transistor device, and the device characteristic of the thin film transistor device is determined according to the transfer curve and the output curve.

In some embodiments of the present disclosure, the device characteristic includes at least one of threshold voltage, device mobility, current on-off ratio, or subthreshold swing.

In some embodiments of the present disclosure, the device characteristic threshold includes at least one of threshold voltage threshold, device mobility threshold, current on-off ratio threshold, or subthreshold swing threshold.

In some embodiments of the present disclosure, the threshold voltage threshold is less than 0.5 V

In some embodiments of the present disclosure, the device mobility threshold is 1 to 1000 cm²/V/s.

In some embodiments of the present disclosure, the current on-off ratio threshold is 10³ to 10⁸.

In some embodiments of the present disclosure, the subthreshold swing threshold is 10 to 300 mV/dec.

In some embodiments of the present disclosure, the step of screening the materials according to a characteristic parameter threshold as first active layer materials further includes storing the first active layer materials in a first database.

In some embodiments of the present disclosure, data in the first database is automatically input and output through an automatic control system.

In some embodiments of the present disclosure, the first active layer database includes characteristic parameters of a type and a material of the active layer material.

In some embodiments of the present disclosure, the step of screening the first active layer materials according to a device characteristic threshold to obtain second active layer materials further includes storing the second active layer materials in a second database.

In some embodiments of the present disclosure, the second active layer database includes characteristic parameters of a type and a material of the active layer material and a device characteristic obtained by simulating the active layer material as the thin film transistor device.

In some embodiments of the present disclosure, data in the first database is automatically input and output through an automatic control system.

In some embodiments of the present disclosure, a method used to calculate the characteristic parameters of the material includes the first principle calculation method.

In some embodiments of the present disclosure, the step of taking the second active layer material as the active layer material of the thin film transistor device to perform an experiment further includes selecting a material of a source electrode and a drain electrode of the thin film transistor device.

The technical solution of the present disclosure will be further described below through specific embodiments in combination with the accompanying drawings. It should be noted that the following specific embodiments are only for illustration, and the protection scope of the present disclosure is not limited to this.

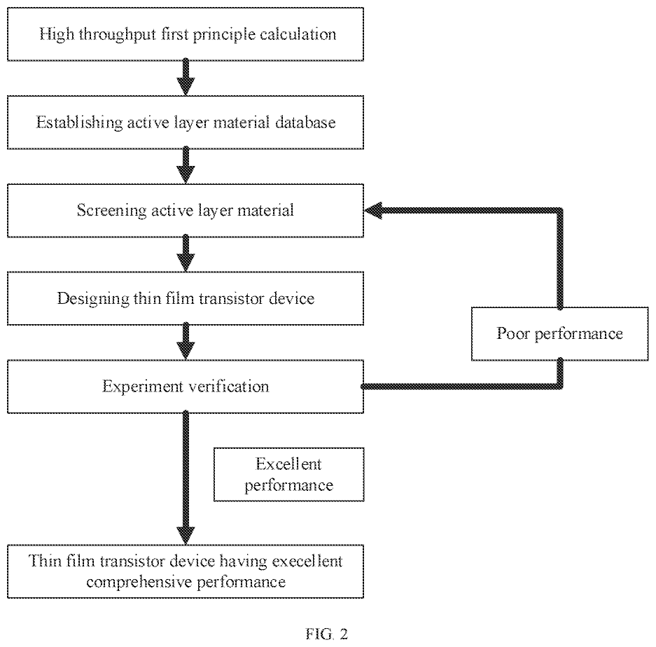

Referring to FIG. 2, the present embodiment provides a method of designing a thin film transistor device, including the following steps:

Step 1: materials possible to become the thin film transistor device, which mainly refers to the active layer material of the thin film transistor, is searched by using the first principle calculation in combination with the high throughput integrated automatic control process; and calculation of energy band structure, band gap, Schottky barrier, work function, and intermediate phase, etc., are mainly performed on the searched active layer materials by using the first principle calculation.

The searched active layer materials include the existing known active layer materials and active layer materials unknown in the prior art. Therefore, the present method may predict active layer materials unknown in the prior art, thereby expanding a selection range of the active layer materials.

All calculation result data will be automatically input and output and will be collected and stored through the high throughput integrated automatic control system (such as MatCloud, MaXFlow); and the high throughput integrated automatic control system is used to achieve automatic input and output as well as collection and storage of data. The high throughput integrated automatic control system is mainly used for data submission, and serves to implement a method of submitting a large number of calculation tasks at one time to assist a user with a series of automatic processes, such as generate recommended calculation parameters from the time when the user starts a material calculation, automatically submit a job, automatically monitor a job status, automatically download a task result, automatically extract an input for a next calculation, generate a final job report, etc. In this way, a calculation process of an entire analog simulation may be achieved fully automatically without manual intervention. On one hand, labor costs may be saved, and on the other hand, incorrect results caused by human extraction may be avoided, so as to ensure a correctness of calculation result extraction.

Step 2: the active layer materials having specific material characteristics in the calculation results are screened out as the first active layer materials, the first active layer materials are analyzed, classified and stored to establish the first database; and the first database includes band structure data, band gap data, the Schottky barriers, the work functions, the possible phase structures, etc. of the active layer materials.

The active layer materials having specific material characteristics in the present embodiment refers to the active layer materials meeting the requirement of characteristic parameter threshold. Specifically, an active layer material meeting the requirement of characteristic parameter threshold is, for example, the active layer material with a band gap range of 0.5 eV-3 eV, a Schottky barrier of 0.1 eV-2 eV, a work function of 2.5 eV-5.5 eV, and an intermediate phase of 1-4. The active layer material meeting the requirement may meet one of the characteristic parameter thresholds, or meet multiple characteristic parameter thresholds. The more characteristic parameter thresholds an active layer material meets, the better performance the active layer material will be.

In the present embodiment, all the data obtained by the theoretical calculation are stored, classified and sorted. In the steps of storing and classifying, different data types are classified into different databases: for example, database A stores the classified energy band structure data, database B stores the classified band gap data, database C stores the classified Schottky barriers, and so on.

Step 3: the active layer materials having a better performance are screened out through a simulation method of establishing a thin film transistor device model based on the established first database.

In this step, the transfer curves and the output curves of the thin film transistor devices constructed by different thin film transistor active layer materials are studied in detail mainly in combination with the device model of the thin film transistor device. The threshold Voltage (Vtn), the device mobility, the current on-off ratio Iₒₙ/I_{off}, the subthreshold swing (SS) of the thin film transistor device are analyzed through the transfer curve and the output curve, and a stability (device characteristics may not change obviously after a long-term operation) and an uniformity of the thin film transistor device are also considered at the same time, and the active layer materials having an excellent comprehensive performance, i.e., the second active layer materials are finally screened out.

The closer the threshold voltage Vₜₕ is to 0, the better. In the present embodiment, the threshold voltage Vₜₕ of the thin film transistor device with the preferred active layer material is selected to be less than 0.5 V. In other embodiments, the threshold voltage Vₜₕ may be, for example, less than 0.4 V, 0.3 V, 0.2 V, 0.1 V, etc.

The larger the value of the device mobility, the better. In the present embodiment, the device mobility of the thin film transistor device with the preferred active layer material is selected to be 1-1000 cm²/N/s. In other embodiments, the device mobility may be 1 cm²N/s, 10 cm²/V/s, 100 cm²/V/s, 200 cm²/V/s, 500 cm²/V/s, 800 cm²/V/s, 1000 cm²/V/s, etc.

In the present embodiment, the current on-off ratio Iₒₙ/I_{off} of the thin film transistor device with the preferred active layer material has a range of 10³-10⁸. When the I_{off} is relatively small, the larger the on-off ratio is, the better. The larger the Iₒₙ, the faster the device operates, and the greater the ability for driving load. The smaller the I_{off}, the lower the power consumption of the device. In other embodiments, the current on-off ratio may be, for example, 10³, 10⁴, 10⁵, 10⁶, 10⁷, and 10⁸.

The smaller a range value of the subthreshold swing, the better. In the present embodiment, the range of the subthreshold swing of the thin film transistor device with the preferred active layer material is selected to be 10-300 mV/dec. In other embodiments, the range of the subthreshold swing may be, for example, 10 mV/dec, 12 mV/dec, 15 mV/dec, 18 mV/dec, 20 mV/dec, 30 mV/dec, 50 mV/dec, 80 mV/dec, 100 mV/dec, 150 mV/dec, 180 mV/dec, 200 mV/dec, 220 mV/dec, 250 mV/dec, 280 mV/dec, 300 mV/dec.

A good stability of the thin film transistor device may be indicated by that, when the device operates for a long time (for example, a range value of 100 hours of continuous operation) and there is no obvious change in the characteristics.

Step 4: the thin film transistor device is designed based on a specific transistor structure (such as a top gate structure or a bottom gate structure) by using the screened out second active layer material of the thin film transistor and selecting a suitable source and drain electrode material. Generally, a selection standard of the source and drain electrodes is to compare the work function of the active layer material and a work function of the electrode material as well as the Schottky barrier between interfaces. The closer the work function of the active layer material and the work function of the electrode material and the smaller the Schottky barrier of the interface formed therebetween, the better.

The source and drain electrodes may be made of at least one of Pt, Au, Cu, Ag, Mo and the like.

Step 5: the thin film transistor device is prepared, the performance of the prepared device is verified, and a design solution is finally adjusted according to characteristic feedback information of the tested thin film transistor device. If the experimental verification result shows that the performance is poor, then the step is returned to screen other types of active layer materials until a thin film transistor device having an excellent comprehensive performance is obtained.

In summary, the method of designing a thin film transistor of the present disclosure has at least one of the following advantages over the prior art:
1. The present disclosure may obtain a large number of physical properties related to active layer materials of a thin film transistor device and the corresponding active layer material database through a simple method, and provide a theoretical guidance for studying the characteristics of the thin film transistor device.
2. The present disclosure provides a simple method of designing a thin film transistor device, thereby saving a lot of resources in the process of developing thin film transistor device.
3. The method of the present disclosure is simple to operate and may be widely applied in the design of various thin film transistor devices with different materials and structures.

The above specific embodiments further describe the objectives, technical solutions and beneficial effects of the present disclosure in detail. It should be understood that the above descriptions are only specific embodiments of the present disclosure, and are not intended to limit the present disclosure. Within the spirit and the principle of the present disclosure, any modifications, equivalent replacements, improvements, etc., shall be included in the protection scope of the present disclosure.

1. A method of designing a thin film transistor device, comprising:
calculating characteristic parameters of searched materials;
screening the materials according to a characteristic parameter threshold to obtain first active layer materials;
simulating the first active layer material as an active layer material in a thin film transistor device model to obtain a device characteristic of the thin film transistor device;
screening the first active layer materials according to a device characteristic threshold to obtain second active layer materials;
taking the second active layer material as the active layer material of the thin film transistor device to perform an experiment; and
selecting another second active layer material to perform the experiment once again when an experiment result does not meet a preset requirement, and a design of the thin film transistor device is completed until the experiment result meets the preset requirement.

2. The method according to claim 1,
wherein the characteristic parameter comprises at least one of energy band structure, band gap, Schottky barrier, work function, and intermediate phase.

3. The method according to claim 1,
wherein the characteristic parameter threshold comprises at least one of band gap threshold, Schottky barrier threshold, work function threshold or intermediate phase threshold.

4. The method according to claim 3,
wherein the band gap threshold is 0.5 to 3 eV;
wherein the Schottky barrier threshold is 0.1 to 2 eV;
wherein the work function threshold is 2.5 to 5.5 eV; and
wherein the intermediate phase threshold is 1 to 4.

5. The method according to claim 1,
wherein the simulating step specifically comprises simulating a transfer curve and an output curve of the thin film transistor device, and determining the device characteristic of the thin film transistor device according to the transfer curve and the output curve.

6. The method according to claim 1,
wherein the device characteristic comprises at least one of threshold voltage, device mobility, current on-off ratio, or subthreshold swing.

7. The method according to claim 1,
wherein the device characteristic threshold comprises at least one of threshold voltage threshold, device mobility threshold, current on-off ratio threshold, or subthreshold swing threshold.

8. The method according to claim 7,
wherein the threshold voltage threshold is less than 0.5 V;
wherein the device mobility threshold is 1 to 1000 cm²/V/s;
wherein the current on-off ratio threshold is 10³ to 10⁸; and
wherein the subthreshold swing threshold is 10 to 300 mV/dec.

9. The method according to claim 1,
wherein the step of screening the materials according to a characteristic parameter threshold as first active layer materials further comprises: storing the first active layer materials in a first database.

10. The method according to claim 9,
wherein data in the first database is automatically input and output through an automatic control system.

11. The method according to claim 9,
wherein the first active layer database comprises characteristic parameters of a type and a material of the active layer material.

12. The method according to claim 1,
wherein the step of screening the first active layer materials according to a device characteristic threshold to obtain second active layer materials further comprises: storing the second active layer materials in a second database.

13. The method according to claim 12,
wherein the second active layer database comprises characteristic parameters of a type and a material of the active layer material and a device characteristic obtained by simulating the active layer material as the thin film transistor device; and
wherein data in the second database is automatically input and output through an automatic control system.

14. The method according to claim 1,
wherein a method used to calculate the characteristic parameter of the material comprises a first principle calculation method.

15. The method according to claim 1,
wherein the step of taking the second active layer material as the active layer material of the thin film transistor device to perform an experiment further comprises: selecting a material of a source electrode and a drain electrode of the thin film transistor device.
